(19) United States
(12) Patent Application Publication
(10) Pub. No.: US 2006/0107869 A1
(43) Pub. Date: May 25, 2006

(54) Inks for digital textile printing with reactive yellow fluorescent dyes

(75) Inventors: Wolfram Reddig, Spartanburg, SC (US); Karl-Hans Blank, Liederbach (DE)

Correspondence Address: CONNOLLY BOVE LODGE & HUTZ, LLP, P O BOX 2207, WILMINGTON, DE, 19899, US

(73) Assignee: DyStar Textilf Deutschland KG, Frankfurt am Main (DE)

(21) Appl. No.: 10/562,497

(22) PCT Filed: Jul. 3, 2004
(86) PCT No.: PCT/EP04/07272

§ 371 Date: Jan. 26, 2006

(30) Foreign Application Priority Data
Jul. 10, 2003 (DE) 103 31 178.5

(51) Int. Cl. C09D 11/00; C07D 491/02

(52) U.S. Cl. 106/31.47; 544/294; 544/209

(57) ABSTRACT

Aqueous textile inkjet printing inks including a reactive fluorescent xanthene dye of the general formula (1) 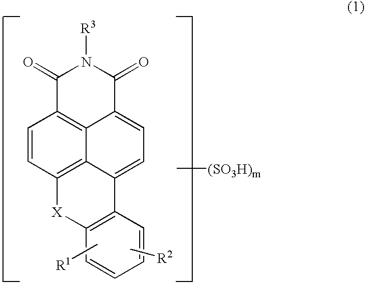 where
R¹, R², R³, X and m are each as defined in claim 1.

Digital printing techniques will become increasingly important in the future in the textile area as well as in the nontextile area.

Changed market expectations in conventional textile printing demand more flexibility in design, color and delivery time. Digital inkjet technology is the answer. By making it possible to print directly from the computer via printing nozzles onto textiles without the need to prepare printing screens, this new technology improves printing process flexibility, efficiency and environmental performance. It provides substantially integrated operations, shortens printing times and meets the demand for rapid reaction to market developments and for fewer intermediate stages in the manufacturing operation.

The inkjet printing process is usually carried out using aqueous inks, which are sprayed as small droplets directly onto the substrate. There is a continuous form of the process, in which the ink is pressed piezoelectrically through a nozzle at a uniform rate and deflected onto the substrate by an electric field, depending on the pattern to be produced, and there is an uninterrupted inkjet or drop-on-demand process, in which the ink is expelled only where a colored dot is to appear. The latter form of the process employs either a piezoelectric crystal or a heated cannula (bubble or thermal jet process) to exert pressure on the ink system and so eject an ink droplet. These techniques are described in Text. Chem. Color, volume 19 (8), pages 23 ff and volume 21, pages 27 ff.

This highly sensitive microtechnology requires the development of tailored dye preparations (inks) meeting, for example, high requirements with regard to purity, particle size, viscosity, surface tension, conductivity, physico-chemical stability, thermophysical properties, the pH, the absence of foam, color strength, fastness level and stability in storage. Commercially available reactive dyes in the form of their powder, granular or liquid formulations of the kind used for conventional, analog textile printing contain significant electrolyte quantities, dustproofing and standardizers which lead to massive problems in inkjet printing. On the other hand, dye inks as used for nontextile materials, for example paper, wood, plastics, ceramics, etc. provide only unsatisfactory results with regard to ease of application and print color yield and fastnesses on textile material. All prior art textile inks rely on chromophores from conventional textile printing which permit the production of relatively bright hues, but do not have fluorescent properties. Fluorescence is needed for special fashion effects as well as for safety clothing.

It is an object of the present invention to provide printing inks which do not have the abovementioned disadvantages.

It has now been found that, surprisingly, inks based on reactive xanthene dyes as known from DE 2 132 963 give excellent results.

The present invention accordingly provides novel aqueous textile inkjet printing inks including a reactive fluorescent xanthene dye of the general formula (1) 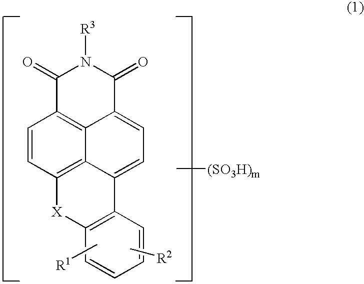
where
R¹ and R² are independently hydrogen atom, halogen atoms, preferably chlorine or bromine atoms, (C₁-C₄)-alkyl- or (C₁-C₄)-alkoxy-,
X is an oxygen or sulfur atom or a CO group,
m is a number from 1-3, and
R³ is a radical of the general formula (2) 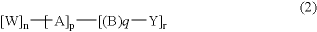
where
W is a bivalent bridge member, such as for example a C₁ to C₄-alkylene,
A is a bivalent mono- or dinuclear aromatic radical, for example a phenylene or naphthylene group or a bivalent diphenyl, diphenyl ether, diphenylamine, diphenyl sulfide or diphenyl sulfone radical and may be substituted in the aromatic nuclei by halogen atoms, preferably chlorine or bromine atoms, lower alkyl groups, lower alkoxy groups, hydroxyl, carboxyl, sulfur or nitro groups,
B is a bivalent bridge member such as for example a C₁ to C₄-alkylene or —NR⁴¹—, wherein R⁴¹ is a hydrogen atom or a lower optionally substituted alkyl radical, such as preferably a methyl, ethyl, β-hydroxyethyl or β-sulfatoethyl group,
Y is a reactor group
n, p, q are 0 or 1, and
r is 1 or 2.

Reactor groups Y are groups which comprise one or more reactive groups or detachable substituents that, on application of the dyes to cellulosic materials in the presence of acid-binding agents with or without heating, are capable of forming covalent bonds with the hydroxyl groups of the cellulose or, on application to superpolyamide fibers, such as wool, are capable of forming covalent bonds with the NH groups of these fibers.

Reactor groups suitable for the purposes of the present invention which contain at least one detachable substituent bound to a heterocyclic radical or to an aliphatic radical include those which contain at least one reactive substituent bound to a 5- or 6-membered heterocyclic optionally substituted ring, such as a monazine, diazine, triazine, for example pyridin, pyrimidine, pyridazine, pyrazine, thiazine, oxazine or asymmetrical or symmetrical triazine ring, or to such a ring system which comprises one or more fused-on aromatic rings, such as a quinoline, phthalazine, cinnoline, quinazoline, quinoxaline, acridine, phenazine and phenanthridine ring system; the 5- or 6-membered heterocyclic rings which comprise at least one reactive substituent are accordingly preferably those which contain one or more nitrogen atoms and may contain fused-on 5- or preferably 6-membered carbocyclic rings. Examples to be mentioned of reactive substituents on the heterocycle are halogen, such as fluorine, chlorine or bromine, ammonium including hydrazinium, sulfonium, sulfonyl, azido, thiocyanato, thio, thioether, oxyether, sulfinic acid and sulfonic acid. Specific examples to be mentioned are 3-chloro- and 3,6-dichloro-1,2-diazinyl radicals, mono- or dihalo-symmetrical-triazinyl radicals, such as for example 2,4-dichloro-6-triazinyl, 2-amino-4-chloro-6-triazinyl, 2-ethylamino- or 2-propylamino-4-chloro-6-triazinyl, 2-β-hydroxyethylamino-4-chloro-6-triazinyl, 2-di(β-hydroxyethyl)amino-4-chloro-6-triazinyl and the corresponding sulfuric monoesters, 2-diethylamino-4-chloro-6-triazinyl, 2-morpholino- or 2-piperidino-4-chloro-6-triazinyl, 2-cyclohexylamino-4-chloro-6-triazinyl, 2-arylamino- and substituted arylamino-4-chloro-6-triazinyl, such as 2-phenylamino-4-chloro-6-triazinyl, 2-(o-, m- or p-carboxy- or sulfophenyl)amino-4-chloro-6-triazinyl, 2-alkoxy-4-chloro-6-triazinyl, such as 2-methoxy- or -ethoxy-4-chloro-6-triazinyl, 2-(phenylsulfonylmethoxy)-4-chloro-6-triazinyl, 2-aryloxy- and substituted aryloxy-4-chloro-6-triazinyl, such as 2-phenoxy-4-chloro-6-triazinyl, 2-(p-sulfophenyl)oxy-4-chloro-6-triazinyl, 2-(o-, m- or p-methyl- or methoxyphenyl)oxy-4-chloro-6-triazinyl, 2-alkylmercapto- or 2-arylmercapto- or 2-(substituted aryl)mercapto-4-chloro-6-triazinyl, such as 2-β-hydroxyethyl)mercapto-4-chloro-6-triazinyl, 2-phenylmercapto-4-chloro-6-triazinyl, 2-(4′-methylphenyl)mercapto-4-chloro-6-triazinyl, 2-(2′,4′-dinitro)phenylmercapto-4-chloro-6-triazinyl, 2-methyl-4-chloro-6-triazinyl, 2-phenyl-4-chloro-6-triazinyl, 2,4,5-trichloro-6-pyrimidinyl, 2,4-dichloro-5-nitro- or -5-methyl- or -5-carboxymethyl- or -5-carboxy- or -5-cyano- or -5-vinyl- or -5-sulfo- or -5-mono, -di- or trichloromethyl- or -5-carboalkoxy-6-pyrimidinyl, 2,6-dichloropyrimidinyl-4-carbonyl, 2,4-dichloropyrimidine-5-carbonyl, 2-chloro-4-methylpyrimidine-5-carbonyl, 2-methyl-4-chloropyrimidine-5-carbonyl, 2-chloro-4-methylpyrimidine-5-carbonyl, 2-methyl-4-chloropyrimidine-5-carbonyl, 2-methylthio-4-fluoropyrimidine-5-carbonyl, 6-methyl-2,4-dichloropyrimidine-5-sulfonyl, 2-chloroquinoxaline-3-carbonyl, 2- or 3-monochloroquinoxaline-6-carbonyl, 2- or 3-monochloroquionxaline-6-sulfonyl, 2,3-dichloroquinoxaline-6-carbonyl, 2,3-dichloroquinoxaline-6-sulfonyl, 1,4-dichlorophthalazine-6-sulfonyl or -6-carbonyl, 2,4-dichloroquinazoline-7- or -6-sulfonyl or -6-carbonyl-, 2- or 3- or 4-(4′,5′-dichloropyridazon-6′-yl-1′)ethylcarbonyl, N-methyl-N-(2,4-dichloro-6-triazinyl)carbamyl, N-methyl-N-(2-methylamino-4-chloro-6-triazinyl)carbamyl, N-methyl-N-(2,4-dichloro-6-triazinyl)carbamyl, N-methyl- or N-ethyl-N-(2,4-dichloro-6-triazinyl)aminoacetyl, N-methyl-N-(2,3-dichloroquinoxaline-6-carbonyl)aminoacetyl, and the corresponding bromine and fluorine derivatives of the abovementioned chloro-substituted heterocyclic radicals, of these, for example 2-fluoro-4-pyrimidinyl, 2,6-difluoro-4-pyrimidinyl, 2,6-difluoro-5-chloro-4-pyrimidinyl, 2-fluoro-5,6-dichloro-4-pyrimidinyl, 2,6-difluoro-5-methyl-4-pyrimidinyl, 2,5-difluoro-6-methyl-4-pyrimidinyl, 2-fluoro-5-methyl-6-chloro-4-pyrimidinyl, 2-fluoro-5-nitro-6-chloro-4-pyrimidinyl, 5-bromo-2-fluoro-4-pyrimidinyl, 2-fluoro-5-cyano-4-pyrimidinyl, 2-fluoro-5-methyl-4-pyrimidinyl, 2,5,6-trifluoro-4-pyrimidinyl, 5-chloro-6-chloromethyl-2-fluoro-4-pyrimidinyl, 2,6-difluoro-5-bromo-4-pyrimidinyl, 2-fluoro-5-bromo-6-methyl-4-pyrimidinyl, 2-fluoro-5-bromo-6-chloromethyl-4-pyrimidinyl, 2,6-difluoro-5-chloromethyl-4-pyrimidinyl, 2,6-difluoro-5-nitro-4-pyrimidinyl, 2-fluoro-6-methyl-4-pyrimidinyl, 2-fluoro-5-chloro-6-methyl-4-pyrimidinyl, 2-fluoro-5-chloro-4-pyrimidinyl, 2-fluoro-6-chloro-4-pyrimidinyl, 6-trifluoromethyl-5-chloro-2-fluoro-4-pyrimidinyl, 6-trifluoromethyl-2-fluoro-4-pyrimidinyl, 2-fluoro-5-nitro-4-pyrimidinyl, 2-fluoro-5-trifluoromethyl-4-pyrimidinyl, 2-fluoro-5-phenyl- or -5-methylsulfonyl-4-pyrimidinyl, 2-fluoro-5-carboxamido-4-pyrimidinyl, 2-fluoro-5-carbomethoxy-4-pyrimidinyl, 2-fluoro-5-bromo-6-trifluoromethyl-4-pyrimidinyl, 2-fluoro-6-carbonamido-4-pyrimidinyl, 2-fluoro-6-cyano-4-pyrimidinyl, 2,6-difluoro-5-methylsulfonyl-4-pyrimidinyl, 2-fluoro-5-sulfonamido-4-pyrimidinyl, 2-fluoro-5-chloro-6-carbomethoxy-4-pyrimidinyl, 2,6-difluoro-5-trifluoromethyl-4-pyrimidinyl; sulfo-containing triazine radicals, such as 2,4-bis(phenylsulfonyl)-6-triazinyl, 2-(3′-carboxyphenyl)sulfonyl-4-chloro-6-triazinyl, 2-(3′-sulfophenyl)sulfonyl-4-chloro-6-triazinyl, 2,4-bis(3′-carboxyphenylsulfonyl-1′)-6-triazinyl; sulfonyl-containing pyrimidine rings, such as 2-carboxymethylsulfonyl-4-pyrimidinyl, 2-methylsulfonyl-6-methyl-4-pyrimidinyl, 2-methylsulfonyl-6-ethyl-4-pyrimidinyl, 2-phenylsulfonyl-5-chloro-6-methyl-4-pyrimidinyl, 2,6-bis(methylsulfonyl)₄-pyrimidinyl, 2,6-bis(methylsulfonyl)-5-chloro-4-pyrimidinyl, 2,4-bis(methylsulfonyl)pyrimidine-5-sulfonyl, 2-methylsulfonyl-4-pyrimidinyl, 2-phenylsulfonyl-4-pyrimidinyl, 2-trichloromethylsulfonyl-6-methyl-4-pyrimidinyl, 2-methylsulfonyl-5-chloro-6-methyl-4-pyrimidinyl, 2-methylsulfonyl-5-bromo-6-methyl-4-pyrimidinyl, 2-methylsulfonyl-5-chloro-6-ethyl-4-pyrimidinyl, 2-methylsulfonyl-5-chloro-6-chloromethyl-4-pyrimidinyl, 2-methylsulfonyl-4-chloro-6-methylpyrimidine-5-sulfonyl, 2-methylsulfonyl-5-nitro-6-methyl-4-pyrimidinyl, 2,5,6-tris(methylsulfonyl)₄-pyrimidinyl, 2-methylsulfonyl-5,6-dimethyl-4-pyrimidinyl, 2-ethylsulfonyl-5-chloro-6-methyl-4-pyrimidinyl, 2-methylsulfonyl-6-chloro-4-pyrimidinyl, 2,6-bis(methylsulfonyl)-5-chloro-4-pyrimidinyl, 2-methylsulfonyl-6-carboxy-4-pyrimidinyl, 2-methylsulfonyl-5-sulfo-4-pyrimidinyl, 2-methylsulfonyl-6-carbomethoxy-4-pyrimidinyl, 2-methylsulfonyl-5-carboxy-4-pyrimidinyl, 2-methylsulfonyl-5-cyano-6-methoxy-4-pyrimidinyl, 2-methylsulfonyl-5-chloro-4-pyrimidinyl, 2-sulfoethylsulfonyl-6-methyl-4-pyrimidinyl, 2-methylsulfonyl-5-bromo-4-pyrimidinyl, 2-phenylsulfonyl-5-chloro-4-pyrimidinyl, 2-carboxymethylsulfonyl-5-chloro-6-methyl-4-pyrimidinyl, 2-methylsulfonyl-6-chloropyrimidine-4- and -5-carbonyl, 2,6-bis(methylsulfonyl)pyrimidine-4- or -5-carbonyl, 2-ethylsulfonyl-6-chloropyrimidine-5-carbonyl, 2,4-bis(methylsulfonyl)pyrimidine-5-sulfonyl, 2-methylsulfonyl-4-chloro-6-methylpyrimidine-5-sulfonyl or -carbonyl; ammonio-containing triazine rings, such as 2-trimethylammonio-4-phenylamino- or -4-(o-, m- or p-sulfophenyl)amino-6-triazinyl, 2-(1,1-dimethylhydrazinio)₄-phenylamino- or 4-(o-, m- or p-sulfophenyl)amino-6-triazinyl, 2-(1,1-dimethylhydrazinio)-4-phenylamino- or 4-(o-, m- or p-sulfophenyl)amino-6-triazinyl, 2-(2-isopropylidine-1,1-dimethyl)hydrazinio-4-phenylamino- or -4-(o-, m- or p-sulfophenyl)amino-6-triazinyl, 2-N-aminopyrrolidinio- or 2-aminopiperidinio-4-phenylamino- or -4-(o-, m- or p-sulfophenyl)triazinyl radicals which contain 1,4-diazabicyclo[2.2.2]octane or 1,2-diazabicyclo[0.3.3]octane bound quaternarily in the 2 position via a nitrogen bond, 2-pyridinio-4-phenylamino- or -4-(o-, m- or p-sulfophenyl)amino-6-triazinyl and the corresponding 2-onio-6-triazinyl radicals which are substituted in the 4 position by alkylamino, such as methylamino, ethylamino or β-hydroxyethylamino, or alkoxy, such as methoxy or ethoxy, or aroxy, such as phenoxy or sulfophenoxy groups; 2- or 3-monochloro- or 2,3-dichloroquinoxaline derivates and the corresponding bromo compounds; 2-chlorobenzothiazole-5- or -5-carbonyl or -5- or -6-sulfonyl, 2-arylsulfonyl or -alkylsulfonyl-5-benzothiazole or -6-carbonyl or -5- or -6-sulfonyl, such as 2-methylsulfonyl- or 2-ethylsulfonylbenzothiazole-5- or -6-sulfonyl- or -carbonyl-, 2-phenylsulfonylbenzothiazole-5- or -6-sulfonyl- or carbonyl- and the corresponding 2-sulfobenzthiazole-5- or -6-carbonyl or -sulfonyl derivatives containing sulfo groups in the fused-on benzene ring, 2-chorobenzoxazole-5- or 6-carbonyl or -sulfonyl, 2-chlorobenzimidazole-5- or 6-carbonyl or sulfonyl, 2-chloro-4-methylthiazole-(1,3)-5-carbonyl- or -4- or -5-sulfonyl, the N-oxide of 4-chloro- or 4-nitroquinoline-5-carbonyl.

Further reactor groups of the aliphatic series are acryloyl, mono-, di- or trichloroacryloyl groups, such as —COCH═CHCl, —CO—CCl═CH₂, —CO—CCl═CH—CH₃, furthermore —CO—CCl═CH—COOH, CO—CH═CCl—COOH, β-chloropropionyl, 3-phenylsulfonylpropionyl, 3-methylsulfonylpropionyl, β-sulfatoethylsulfonyl, -vinylsulfonyl, β-phosphatoethylsulfonyl, β-methylsulfonylethylsulfonyl, β-phenylsulfonylethylsulfonyl, 2-fluoro-2-chloro-3,3-difluorocyclobutane-1-carbonyl, 2,2,3,3-tetrafluorocyclobutyl-1-acryloyl, β-(2,2,3,3-tetrafluoro-4-methylcyclobutyl-1-)acryloyl, α- or β-bromoacryloyl, α- or β-alkyl or arylsulfonylacryloyl groups such as α- or β-methylsulfonylacryloyl and β-chloroethylsulfonyl[2.2.1]bicycloheptyl.

In preferred dyes of the general formula (1) Y is a reactor group of the general formula (a) to (d): 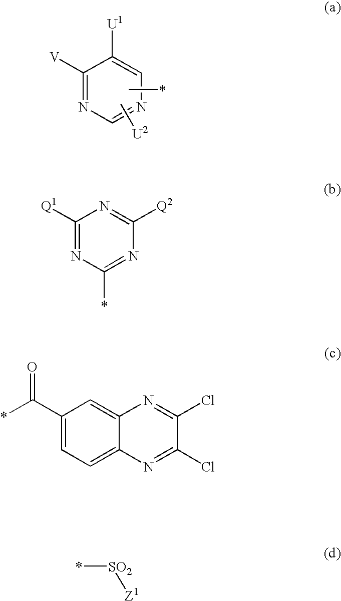
where
V is fluorine or chlorine;
U¹, U² are independently fluorine, chlorine or hydrogen; and
Q¹, Q² are independently chlorine, fluorine, cyanamide, hydroxyl, (C₁-C₆)-alkoxy, phenoxy, sulfophenoxy, mercapto, (C₁-C₆)-alkylmercapto, pyridino, carboxypyridino, carbamoylpyridino or a group of the general formula (7) or (8) 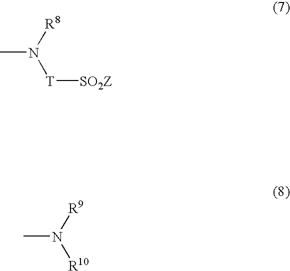
where
   R⁸ is hydrogen or (C₁-C₆)-alkyl, sulfo-(C₁-C₆)-alkyl or phenyl which is unsubstituted or substituted by (C₁-C₄)-alkyl, (C₁-C₄)-alkoxy, sulfur, halogen, carboxyl, acetamido, ureido;
   R⁹ and R¹⁰ independently have one of the meanings of R⁸ or combine to form a cyclic ring system of the formula —(CH₂)ⱼ—, wherein j is 4 or 5, or alternatively —(CH₂)₂-E-(CH₂)₂—, wherein E is oxygen, sulfur, sulfonyl, —NR¹¹ where R¹¹=(C₁-C₆)-alkyl;
   T is phenylene, which is unsubstituted or substituted by 1 or 2 substituents, such as (C₁-C₄)-alkyl, (C₁-C₄)-alkoxy, carboxyl, sulfur, chlorine, bromine, or is (C₁-C₄)-alkylenearylene or (C₂-C₆)-alkylene, which may be interrupted by oxygen, sulfur, sulfonyl, amino, carbonyl, carboxamido, or is phenylene-CONH-phenylene which is unsubstituted or substituted by (C₁-C₄)-alkyl, (C₁-C₄)-alkoxy, hydroxyl, sulfur, carboxyl, amido, ureido or halogen, or is naphthylene which is unsubstituted or substituted by one or two sulfur groups; and
   Z¹ and Z denotes —CH═CH₂, —CH₂CH₂Z² or hydroxyl,
   where
      Z² is hydroxyl or an alkali-detachable group.

The reative dyes used are known and are obtainable by customary diazotization, coupling and condensation reactions.

The printing inks of the present invention include one or more of the reactive dyes mentioned, for example in amounts from 0.1% by weight to 50% by weight, preferably in amounts from 1% by weight to 30% by weight and more preferably in amounts from 1% by weight to 15% by weight based on the total weight of the ink. They may likewise include combinations of the fluorescent reactive dyes mentioned with other reactive dyes used in textile printing.

For the inks to be used in the continuous flow process, a conductivity of 0.5 to 25 mS/m can be set by adding an electrolyte.

Useful electrolytes include for example lithium nitrate and potassium nitrate.

The dye inks of the present invention may include organic solvents at a total level of 1-50% and preferably 5-30% by weight.

Suitable organic solvents are for example
alcohols, for example methanol, ethanol, 1-propanol, isopropanol, 1-butanol, tert-butanol, pentyl alcohol,
polyhydric alcohols for example: 1,2-ethanediol, 1,2,3-propanetriol, butanediol, 1,3-butanediol, 1,4-butanediol, 1,2-propanediol, 2,3-propanediol, pentanediol, 1,4-pentanediol, 1,5-pentanediol, hexanediol, D,L-1,2-hexanediol, 1,6-hexanediol, 1,2,6-hexanetriol, 1,2-octanediol,
polyalkylene glycols, for example: polyethylene glycol, polypropylene glycol, alkylene glycols having 2 to 8 alkylene groups, for example monoethylene glycol, diethylene glycol, triethylene glycol, tetraethylene glycol, thioglycol, thiodiglycol, butyltriglycol, hexylene glycol, propylene glycol, dipropylene glycol, tripropylene glycol,
low alkyl ethers of polyhydric alcohols, for example: ethylene glycol monomethyl ether, ethylene glycol monoethyl ether, ethylene glycol monobutyl ether, diethylene glycol monomethyl ether, diethylene glycol monoethyl ether, diethylene glycol monobutyl ether, diethylene glycol monohexyl ether, triethylene glycol monomethyl ether, triethylene glycol monobutyl ether, tripropylene glycol monomethyl ether, tetraethylene glycol monomethyl ether, tetraethylene glycol monobutyl ether, tetraethylene glycol dimethyl ether, propylene glycol monomethyl ether, propylene glycol monoethyl ether, propylene glycol monobutyl ether, tripropylene glycol isopropyl ether, polyalkylene glycol ethers, such as for example: polyethylene glycol monomethyl ether, polypropylene glycol glycerol ether, polyethylene glycol tridecyl ether, polyethylene glycol nonylphenyl ether,
amines, such as, for example: methylamine, ethylamine, triethylamine, diethylamine, dimethylamine, trimethylamine, dibutylamine, diethanolamine, triethanolamine, N-acetylethanolamine, N-formylethanolamine, ethylenediamine,
urea derivatives, such for example: urea, thiourea, N-methylurea, N,N′-epsilon-dimethylurea, ethyleneurea, 1,1,3,3-tetramethylurea,
amides, such as for example: dimethylformamide, dimethylacetamide, acetamide,
ketones or keto alcohols, such as for example: acetone, diacetone alcohol,
cyclic ethers, such as for example; tetrahydrofuran, trimethylolethane, trimethylolpropane, 2-butoxyethanol, benzyl alcohol, 2-butoxyethanol, gamma butyrolactone, epsilon-caprolactam
further sulfolane, dimethylsulfolane, methylsulfolane, 2,4-dimethylsulfolane, dimethyl sulfone, butadiene sulfone, dimethyl sulfoxide, dibutyl sulfoxide, N-cyclohexylpyrrolidone, N-methyl-2-pyrrolidone, N-ethylpyrrolidone, 2-pyrrolidone, 1-(2-hydroxyethyl)-2-pyrrolidone, 1-(3-hydroxypropyl)-2-pyrrolidone, 1,3-dimethyl-2-imidazolidinone, 1,3-dimethyl-2-imidazolinone, 1,3-bismethoxymethylimidazolidine, 2-(2-methoxyethoxy)ethanol, 2-(2-ethoxyethoxy)ethanol, 2-(2-butoxyethoxy)ethanol, 2-(2-propoxyethoxy)ethanol, pyridine, piperidine, butyrolactone, trimethylpropane, 1,2-dimethoxypropane, dioxane, ethyl acetate, ethylenediaminetetraacetate, ethyl pentyl ether, 1,2-dimethoxypropane, trimethylolpropane.

The printing inks of the invention may further include customary additives, for example viscosity moderators to set viscosities in the range from 1.5 to 40.0 mPa*s in a temperature range from 20 to 50° C. Preferred inks have a viscosity of 1.5 to 20 mPa*s and particularly preferred inks have a viscosity of 1.5 to 15 mPa*s.

Useful viscosity moderators include rheological additives, for example:

polyvinylcaprolactam, polyvinylpyrrolidone and their copolymers polyetherpolyol, associative thickeners, polyurea, polyurethane, sodium alginates, modified galactomannans, polyetherurea, polyurethane, nonionic cellulose ethers.

As further additives the inks of the invention may include surface-active substances to set surface tensions of 20 to 65 mN/m, which are adapted if necessary as a function of the process used (thermal or piezotechnology).

Useful surface-active substances include for example:

nonionic surfactants, butyldiglycol, 1,2-hexanediol.

The inks may further include customary additives, for example substances to inhibit fungal and bacterial growth in amounts of 0.01 to 1% by weight based on the total weight of the ink.

The inks may be prepared in a conventional manner by mixing the components in water.

The dye inks of the invention are useful in inkjet printing processes for printing a wide variety of pretreated materials, such as silk, leather, wool, polyamide fibers and polyurethanes, and especially cellulosic fiber materials of any kind. Such fiber materials are for example the natural fiber cellulose fibers, such as cotton, linen and hemp, and also pulp and regenerated cellulose. The printing inks of the invention are also useful for printing pretreated hydroxyl- or amino-containing fibers present in blend fabrics, for example blends of cotton, silk, wool with polyester fibers or polyamide fibers.

In contrast to conventional textile printing, where the printing ink already contains all the fixing chemicals and thickeners for a reactive dye, in inkjet printing the auxiliaries have to be applied to the textile substrate in a separate pretreatment step.

The pretreatment of the textile substrate, for example cellulose and regenerated cellulose fibers and also silk and wool, is effected with an aqueous alkaline liquor prior to printing. To fix reactive dyes there is a need for alkali, for example sodium carbonate, sodium bicarbonate, sodium acetate, trisodium phosphate, sodium silicate, sodium hydroxide, alkali donors such as, for example, sodium chloroacetate, sodium formate, hydrotropic substances such as, for example, urea, reduction inhibitors, for example sodium nitrobenzenesulfonates, and also thickeners to prevent flowing of the motives when the printing ink is applied, for example sodium alginates, modified polyacrylates or highly etherified galactomannans.

These pretreatment reagents are uniformly applied to the textile substrate in a defined amount using suitable applicators, for example using a 2- or 3-roll pad, contactless spraying technologies, by means of foam application or using appropriately adapted inkjet technologies, and subsequently dried.

After printing, the textile fiber material is dried at 120 to 150° C. and subsequently fixed.

The fixing of the inkjet prints prepared with reactive dyes may be effected at room temperature or with saturated steam, with superheated steam, with hot air, with microwaves, with infrared radiation, with laser or electron beams or with other suitable energy transfer techniques.

A distinction is made between one- and two-phase fixing processes:

In one-phase fixing, the necessary fixing chemicals are already on the textile substrate.

In two-phase fixing, this pretreatment is unnecessary. Fixing only requires alkali, which, following inkjet printing, is applied prior to the fixing process, without intermediate drying. There is no need for further additives such as urea or thickener.

Fixing is followed by the print aftertreatment, which is the prerequisite for good fastnesses, high brilliance and an impeccable white ground.

The prints prepared with the dye inks of the invention, especially on cellulose fiber materials, have high color strength and a high fiber-dye bond stability not only in the acidic but also in the alkaline range, good lightfastness and very good wetfastness properties, such as wash, water, seawater, cross-dyeing and perspiration fastnesses, and also good fastness to heat setting and pleating and crockfastness.

The examples which follow illustrate the invention. Parts and percentages are by weight, unless otherwise stated. Parts by weight relate to parts by volume as the kilogram relates to the liter.

### EXAMPLE 1

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%.

The thus pretreated textile is printed with an aqueous ink including
2% of dye (3) 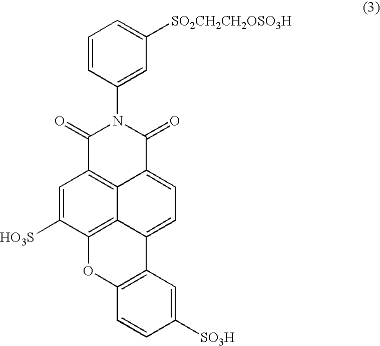
20% of sulfolane,
0.01% of Mergal K9N,
77.99% of water
using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 2

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%.

The thus pretreated textile is printed with an aqueous ink including
5% of dye (4) 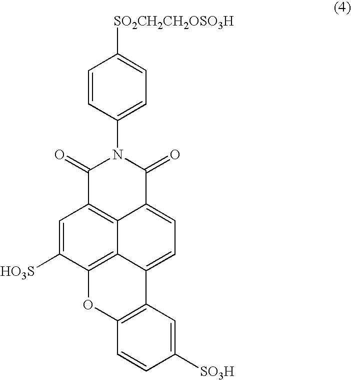
20% of sulfolane,
0.01% of Mergal K9N,
74.99% of water
using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 3

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%.

The thus pretreated textile is printed with an aqueous ink including
3% of dye (5) 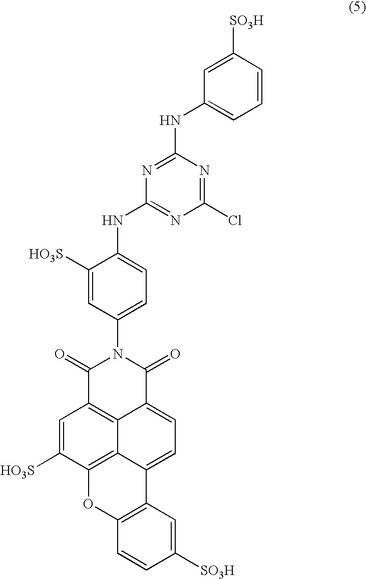
15% of sulfolane,
0.01% of Mergal K9N,
81.99% of water
using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 4

A textile fabric of causticized viscose is padded with liquor containing 30 g/l of anhydrous sodium carbonate, 200 g/l of urea and 150 g/l of a low viscosity sodium alginate solution (6%) and then dried. The wet pickup is 70%.

The thus pretreated textile is printed with an aqueous ink including
4% of dye (6) 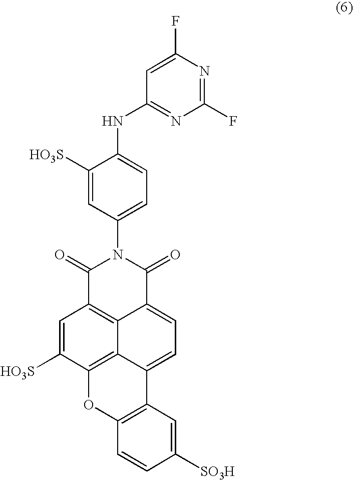
18% of sulfolane,
0.01% of Mergal K9N,
77.99% of water
using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 5

A textile fabric of mercerized cotton is padded with liquor containing 35 g/l of anhydrous sodium carbonate, 50 g/l of urea and 150 g/l of a low viscosity sodium alginate solution (6%) and then dried. The wet pickup is 70%.

The thus pretreated textile is printed with an aqueous ink including

8% of dye (3)

20% of 1,2-propanediol

0.01% of Mergal K9N and

71.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried. The result is a very bright greenish yellow print having excellent use fastnesses.

### EXAMPLE 6

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

8% of dye (3)

15% of N-methylpyrrolidone

0.01% of Mergal K9N and

77.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright greenish yellow print having excellent use fastnesses.

### EXAMPLE 7

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

1% of dye (3)

17% of dipropylene glycol

0.01% of Mergal K9N and

81.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 8

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

3% of dye (3)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright greenish yellow print having excellent use fastnesses.

### EXAMPLE 9

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%.

The thus pretreated textile is printed with an aqueous ink including

3% of dye (3)

20% of sulfolane

10% of urea

0.01% of Mergal K9N,

66.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright greenish yellow print having excellent use fastnesses.

### EXAMPLE 10

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%.

The thus pretreated textile is printed with an aqueous ink including

5% of dye (3)

15% of 1,2-propanediol

10% of urea

0.01% of Mergal K9N, and

66.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried. The result is a very bright yellow print having excellent use fastnesses.

### EXAMPLE 11

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

3% of dye (3)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes.

The print is then rinsed warm in the presence of 1-2 ml of 25% ammonia, subjected to a fastness wash with hot water at 70 to 80° C., rinsed warm and then dried.

The result is a very bright yellow print having excellent use fastnesses.

### EXAMPLE 12

A textile fabric of degummed silk is padded with a liquor containing 50 g/l of sodium acetate, 100 g/l of urea and 150 g/l of a highly etherified galactomannan (5%) and then dried. The wet pickup is 70%.

The thus pretreated textile is printed with an aqueous ink containing

5% of dye (3)

15% of 1,2-propanediol

10% of urea

0.01% of Mergal K9N and

69.99% of water

using a drop-on-demand (bubblejet) inkjet print head. The print is fully dried. It is fixed by means of saturated steam at 102° C. for 8 minutes.

The print is then rinsed warm in the presence of 1-2 ml of 25% ammonia, subjected to a fastness wash with hot water at 70 to 80° C., rinsed warm and then dried.

The result is a very bright yellow print having excellent use fastnesses.

### EXAMPLE 13

A textile fabric of mercerized cotton is padded with liquor containing 35 g/l of anhydrous sodium carbonate, 50 g/l of urea and 150 g/l of a low viscosity sodium alginate solution (6%) and then dried. The wet pickup is 70%.

The thus pretreated textile is printed with an aqueous ink including

8% of dye (3)

20% of 1,2-propanediol

0.25% of Leonil SR

0.01% of Mergal K9N and

71.74% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 14

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%.

The thus pretreated textile is printed with an aqueous ink including

8% of dye (3)

15% of 1,2-propanediol

0.01% of Mergal K9N and

77.99% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 15

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

3% of dye (3)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright yellow print having excellent use fastnesses.

### EXAMPLE 16

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

5% of dye (3)

15% of 1,2-propanediol

10% of urea

0.25% of Leonil SR

0.01% of Mergal K9N and

69.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright yellow print having excellent use fastnesses.

### EXAMPLE 17

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

10% of dye (3)

17% of dipropylene glycol

10% of urea

0.25% of Leonil SR

0.01% of Mergal K9N and

62.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 18

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

8% of dye (3)

15% of 1,2-hexanediol

0.01% of Mergal K9N, and

77.99% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 19

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

3% of dye (3)

20% of sulfolane

10% of urea

0.25% of Leonil SR

0.01% of Mergal K9N, and

66.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright yellow print having excellent use fastnesses.

### EXAMPLE 20

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

8% of dye (3)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to 80° C., rinsed warm and then dried.

The result is a very bright greenish yellow print having excellent use fastnesses.

### EXAMPLE 21

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

3% of dye (3)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to 80° C., rinsed warm and then dried.

The result is a very bright yellow print having excellent use fastnesses.

### EXAMPLE 22

A textile fabric of mercerized cotton is padded with liquor containing 35 g/l of anhydrous sodium carbonate, 50 g/l of urea and 150 g/l of a low viscosity sodium alginate solution (6%) and then dried. The wet pickup is 70%.

The thus pretreated textile is printed with an aqueous ink including

8% of dye (4)

20% of 1,2-propanediol

0.01% of Mergal K9N and

71.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 23

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%.

The thus pretreated textile is printed with an aqueous ink including

8% of dye (4)

15% of N-methylpyrrolidone

0.01% of Mergal K9N and

77.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 24

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

1% of dye (4)

17% of dipropylene glycol

0.01% of Mergal K9N and

81.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 25

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%.

The thus pretreated textile is printed with an aqueous ink including

3% of dye (4)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright yellow print having excellent use fastnesses.

### EXAMPLE 26

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%.

The thus pretreated textile is printed with an aqueous ink including

8% of dye (4)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 27

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%.

The thus pretreated textile is printed with an aqueous ink including

1% of dye (4)

17% of dipropylene glycol

0.25% of Leonil SR

0.01% of Mergal K9N and

81.74% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 28

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%.

The thus pretreated textile is printed with an aqueous ink including

3% of dye (4)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright yellow print having excellent use fastnesses.

### EXAMPLE 29

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%.

The thus pretreated textile is printed with an aqueous ink including

8% of dye (4)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright greenish yellow print having excellent use fastnesses.

### EXAMPLE 30

A textile fabric of degummed silk is padded with a liquor containing 50 g/l of sodium acetate, 100 g/l of urea and 150 g/l of a highly etherified galactomannan (5%) and then dried. The wet pickup is 70%.

The thus pretreated textile is printed with an aqueous ink containing

8% of dye (4)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes.

The print is then rinsed warm in the presence of 1-2 ml of 25% ammonia, subjected to a fastness wash with hot water at 70 to 80° C., rinsed warm and then dried.

The result is a very bright greenish yellow print having excellent use fastnesses.

### EXAMPLE 31

A textile fabric of degummed silk is padded with a liquor containing 50 g/l of sodium acetate, 100 g/l of urea and 150 g/l of a highly etherified galactomannan (5%) and then dried. The wet pickup is 70%.

The thus pretreated textile is printed with an aqueous ink containing

3% of dye (4)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to 80° C., rinsed warm and then dried. The result is a very bright yellow print having excellent use fastnesses.

### EXAMPLE 32

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

3% of dye (5)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright yellow print having excellent use fastnesses.

### EXAMPLE 33

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

3% of dye (5)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright yellow print having excellent use fastnesses.

### EXAMPLE 34

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

3% of dye (5)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to 80° C., rinsed warm and then dried. The result is a very bright yellow print having excellent use fastnesses.

### EXAMPLE 35

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

8% of dye (5)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 36

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%.

The thus pretreated textile is printed with an aqueous ink including

3% of dye (5)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright yellow print having excellent use fastnesses.

### EXAMPLE 37

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

5% of dye (5)

15% of 1,2-propanediol

10% of urea

0.25% of Leonil SR

0.01% of Mergal K9N and

69.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright yellow print having excellent use fastnesses.

### EXAMPLE 38

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

10% of dye (5)

17% of dipropylene glycol

10% of urea

0.25% of Leonil SR

0.01% of Mergal K9N and

62.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 39

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

8% of dye (5)

15% of hexanediol

0.01% of Mergal K9N and

77.99% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 40

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

3% of dye (5)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright yellow print having excellent use fastnesses.

### EXAMPLE 41

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

8% of dye (5)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to 80° C., rinsed warm and then dried.

The result is a very bright greenish yellow print having excellent use fastnesses.

### EXAMPLE 42

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

3% of dye (5)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to ° DEG C., rinsed warm and then dried.

The result is a very bright yellow print having excellent use fastnesses.

### EXAMPLE 43

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

3% of dye (6)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright yellow print having excellent use fastnesses.

### EXAMPLE 44

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

5% of dye (6)

15% of 1,2-propanediol

10% of urea

0.01% of Mergal K9N and

69.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright yellow print having excellent use fastnesses.

### EXAMPLE 45

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%.

The thus pretreated textile is printed with an aqueous ink including

10% of dye (6)

17% of dipropylene glycol

10% of urea

0.01% of Mergal K9N and

62.99% of water

using a drop-on-demand (bubblejet) inkjet print head. The print is fully dried. It is fixed by means of saturated steam at 102° C. for 8 minutes. The print is then rinsed warm, subjected to a fastness wash with hot water at 95° C., rinsed warm and then dried. The result is a very bright greenish yellow print having excellent use fastnesses.

### EXAMPLE 46

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

3% of dye (6)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright yellow print having excellent use fastnesses.

### EXAMPLE 47

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

3% of dye (6)

20% of sulfolane

10% of urea

0.01% of Mergal K9N and

66.99% of water

using a drop-on-demand (bubblejet) inkjet print head. The print is fully dried. It is fixed by means of saturated steam at 102° C. for 8 minutes.

The print is then rinsed warm in the presence of 1-2 ml of 25% ammonia, subjected to a fastness wash with hot water at 70 to 80° C., rinsed warm and then dried.

The result is a very bright yellow print having excellent use fastnesses.

### EXAMPLE 48

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

8% of dye (6)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 49

A textile fabric of mercerized cotton is padded with liquor containing 35 g/l of anhydrous sodium carbonate, 100 g/l of urea and 150 g/l of a low viscosity sodium alginate solution (6%) and then dried. The wet pickup is 70%. The thus pretreated textile is printed with an aqueous ink including

1% of dye (6)

17% of dipropylene glycol

0.25% of Leonil SR

0.01% of Mergal K9N and

81.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 50

A textile fabric of mercerized cotton is padded with liquor containing 35 g/l of anhydrous sodium carbonate, 150 g/l of urea and 150 g/l of a low viscosity sodium alginate solution (6%) and then dried. The wet pickup is 70%. The thus pretreated textile is printed with an aqueous ink including

3% of dye (6)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright yellow print having excellent use fastnesses.

### EXAMPLE 51

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

8% of dye (6)

15% of 1,2-hexanediol

0.01% of Mergal K9N and

7.99% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright greenish yellow print having excellent use fastnesses.

### EXAMPLE 52

A textile fabric of causticized viscose is padded with liquor containing 35 g/l of anhydrous sodium carbonate, 200 g/l of urea and 150 g/l of a low viscosity sodium alginate solution (6%) and then dried. The wet pickup is 70%. The thus pretreated textile is printed with an aqueous ink including

3% of dye (6)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm, subjected to a fastness wash with hot water at 95° C., rinsed warm and then dried.

The result is a very bright yellow print having excellent use fastnesses.

### EXAMPLE 53

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

8% of dye (6)

15% of 1,2-hexanediol

0.01% of Mergal K9N and

77.99% of water

using a drop-on-demand (piezo) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to 80° C., rinsed warm and then dried. The result is a very bright greenish yellow print having excellent use fastnesses.

### EXAMPLE 54

A textile fabric of degummed silk is padded with a liquor containing 50 g/l of sodium acetate, 100 g/l of urea and 150 g/l of a highly etherified galactomannan (5%) and then dried. The wet pickup is 70%. The thus pretreated textile is printed with an aqueous ink containing

3% of dye (6)

20% of sulfolane

10% of urea

0.25% of Leonil SR

0.01% of Mergal K9N and

66.74% of water

using a drop-on-demand (piezo) inkjet print head. The print is fully dried. It is fixed by means of saturated steam at 102° C. for 8 minutes. The print is then rinsed warm in the presence of 1-2 ml of 25% ammonia, subjected to a fastness wash with hot water at 70 to 80° C., rinsed warm and then dried. The result is a very bright yellow print having excellent use fastnesses.

### EXAMPLE 55

A textile fabric of mercerized cotton is padded with liquor containing 40 g/l of sodium bicarbonate, 100 μl of urea and 150 g/l of a low viscosity sodium alginate solution (6%) and then dried. The wet pickup is 70%. The thus pretreated textile is printed with an aqueous ink including

5% of dye (5)

0.5% of C. I. Reactive Blue 72

10% of 1,2-hexanediol

20% of sulfolane

10% of urea

0.01% of Mergal K9N and

64.49% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright yellowish green print having excellent use fastnesses.

### EXAMPLE 56

A textile fabric of mercerized cotton is padded with liquor containing 40 g/l of sodium bicarbonate, 100 g/l of urea and 150 g/l of a low viscosity sodium alginate solution (6%) and then dried. The wet pickup is 70%.

The thus pretreated textile is printed with an aqueous ink including

6% of C. I. dye (3)

5% of C. I. Reactive Blue 72

15% of sulfolane

10% of 1,2-hexanediol

10% of dipropylene glycol

0.01% of Mergal K9N and

53.99% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright yellowish green print having excellent use fastnesses.

### EXAMPLE 57

A textile fabric of mercerized cotton is padded with liquor containing 35 g/l of anhydrous sodium carbonate, 50 g/l of urea and 150 g/l of a low viscosity sodium alginate solution (6%) and then dried. The wet pickup is 70%.

The thus pretreated textile is printed with an aqueous ink including

15% of dye (5)

1% of C. I. Reactive Orange 13

10% of 1,2-hexanediol

20% of sulfolane

0.01% of Mergal K9N and

53.99% of water

using a drop-on-demand (piezo) inkjet print head. The print is fully dried. It is fixed by means of saturated steam at 102° C. for 8 minutes.

The print is then rinsed warm, subjected to a fastness wash with hot water at 95° C., rinsed warm and then dried.

The result is a very bright golden yellow print having excellent use fastnesses.

1. Aqueous textile inkjet printing inks including a reactive fluorescent xanthene dye of the general formula (1) 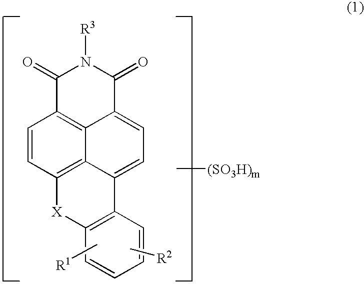
where
R¹ and R² are independently hydrogen, halogen, (C₁-C₄)-alkyl- or (C₁-C₄)-alkoxy-,
X is an oxygen or sulfur atom or a CO group,
m is a number from 1-3 and
R³ is a radical of the general formula (2) 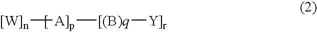
where
W is a bivalent bridge member,
A is a bivalent mono- or dinuclear substituted or unsubstituted aromatic radical
B is a C₁ to C₄-alkylene- or —NR⁴¹—, wherein R⁴¹ is a hydrogen atom or a lower optionally substituted alkyl radical,
Y is a reactor group
n, p, q are 0 or 1, and
r is 1 or 2.

2. An aqueous textile inkjet printing ink including a reactive fluorescent xanthene dye of the general formula (1) as per claim 1, wherein in the formula (2)
W is a C₁ to C₄-alkylene,
B is a C₁ to C₄-alkylene- or —NR⁴¹—, wherein R⁴¹ is a hydrogen atom or a lower optionally substituted alkyl radical,
A is an unsubstituted or substituted phenylene, naphthylene or diphenylene radical, and
Y is a reactor group of the general formula (a) to (d) 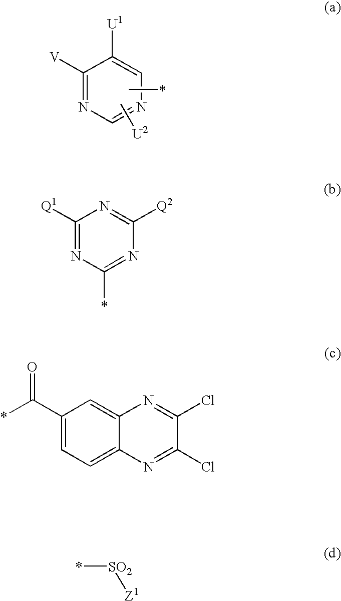
where
V is fluorine or chlorine;
U¹ and U² are independently fluorine, chlorine or hydrogen; and
Q¹ and Q² are independently chlorine, fluorine, cyanamido, hydroxyl, (C₁-C₆)-alkoxy, phenoxy, sulfophenoxy, mercapto, (C₁-C₆)-alkylmercapto, pyridino, carboxypyridino, carbamoylpyridino or a group of the general formula (7) or (8) 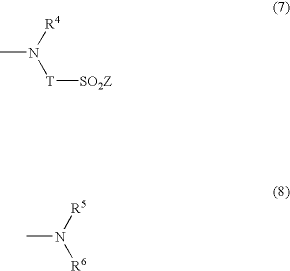
where
R⁴ is hydrogen or (C₁-C₆)-alkyl, sulfo-(C₁-C₆)-alkyl or phenyl which is unsubstituted or substituted by (C₁-C₄)-alkyl, (C₁-C₄)-alkoxy, sulfur, halogen, carboxyl, acetamido or ureido;
R⁵ and R⁶ independently have one of the meanings of R⁴ or combine to form a cyclic ring system of the formula —(CH₂)ⱼ—, wherein j is 4 or 5, or alternatively —(CH₂)₂-E-(CH₂)₂—, wherein E is oxygen, sulfur, sulfonyl, —NR⁷ where R⁷═(C₁-C₆)-alkyl;
T is phenylene, which is unsubstituted or substituted by 1 or 2 substituents, or is (C₁-C₄)-alkylenearylene or (C₂-C₆)-alkylene, which is optionally interrupted by oxygen, sulfur, sulfonyl, amino, carbonyl, carboxamido, or is phenylene-CONH-phenylene which is unsubstituted or substituted by (C₁-C₄)-alkyl, (C₁-C₄)-alkoxy, hydroxyl, sulfur, carboxyl, amido, ureido or halogen, or is naphthylene which is unsubstituted or substituted by one or two sulfur groups; and
Z¹ and Z denotes —CH═CH₂, —CH₂CH₂Z² or hydroxyl,
where
Z² is hydroxyl or an alkali-detachable group.

3. An aqueous textile inkjet printing ink including a reactive fluorescent xanthene dye of the general formula (1) as per claim 1, wherein in the formula (2)
n and p are 0 and
Y is a group of the general formula (d).

4. An aqueous textile inkjet printing ink including a reactive fluorescent xanthene dye of the general formula (1) as per claim 1, wherein in the formula (2)
n is 0,
A is a substituted phenylene radical and
Y is a group of the general formula (a) to (c).

5. An aqueous textile inkjet printing ink including a reactive fluorescent xanthene dye of the general formula (1) as per claim 1, wherein in the formula (2)
n is 0,
A is sulfophenylene and
Y is a group of the general formula (d).

6. An aqueous textile inkjet printing ink including a reactive fluorescent xanthene dye of the general formula (1) as per claim 1, wherein in the formula (2)
n is 0,
p is 1,
m is 2,
X is oxygen,
R¹ is methoxy or hydrogen,
A is phenylene and
Y is a group of the general formula (d).

7. Aqueous textile inkjet printing inks which comprises a reactive fluorescent xanthene dye of the formula (5)

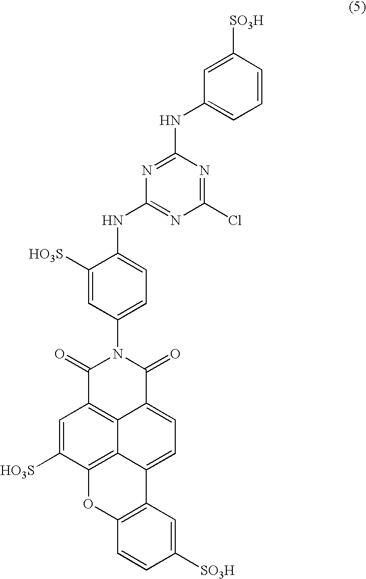

8. Aqueous textile inkjet printing inks which comprises a reactive fluorescent xanthene dye of the formula (6)

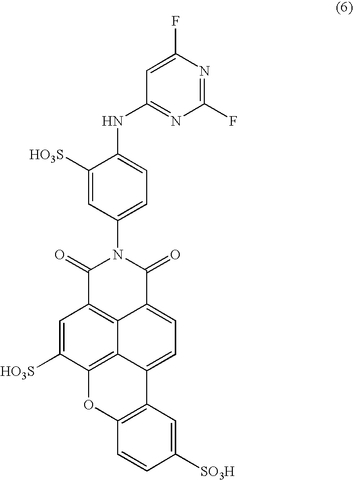

9. Aqueous textile inkjet printing inks which comprises a reactive fluorescent xanthene dye of the formula (3)

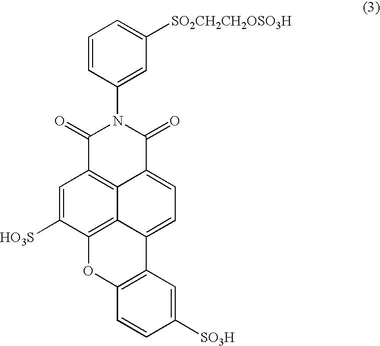

10. Aqueous printing inks as per claim 1 for textile printing by the inkjet process which include one or more reactive dyes of the general formula (1) in amounts from 0.01% by weight to 40% by weight based on the total weight of the inks.

11. Aqueous textile inkjet printing inks as per claim 1 which include 1% to 40% of organic solvents based on the total weight of the ink.

12. A process for printing textile fiber materials by the inkjet process, which comprises utilizing the printing ink as per claim 1.

13. The printing ink as claimed in claim 2, wherein T is phenylene, which is unsubstituted or substituted by 1 or 2 substituents, selected from the group consisting of (C₁-C₄)-alkyl, (C₁-C₄)-alkoxy, carboxyl, sulfur, chlorine and bromine.

14. Aqueous textile inkjet printing inks as per claim 7 which further comprises 1% to 40% of organic solvents based on the total weight of the ink.

15. A process for printing textile fiber materials by the inkjet process, which comprises utilizing the printing ink as per claim 7.

16. Aqueous textile inkjet printing inks as per claim 8 which further comprises 1% to 40% of organic solvents based on the total weight of the ink.

17. A process for printing textile fiber materials by the inkjet process, which comprises utilizing the printing ink as per claim 8.

18. Aqueous textile inkjet printing inks as per claim 9 which further comprises 1% to 40% of organic solvents based on the total weight of the ink.

19. A process for printing textile fiber materials by the inkjet process, which comprises utilizing the printing ink as per claim 9.
